(19) United States
(12) Patent Application Publication
(10) Pub. No.: US 2001/0000801 A1
(43) Pub. Date: May 3, 2001

(54) Hydrophilic sleeve

(76) Inventors: Paul J. Miller, St. Paul, MN (US); Dachuan Yang, Plymouth, MN (US)

Correspondence Address: VIDAS, ARRETT & STEINKRAUS, P.A., 6109 BLUE CIRCLE DRIVE, SUITE 2000, MINNETONKA, MN, 55343-9185, US

(21) Appl. No.: 09/745,628

(22) Filed: Dec. 21, 2000

Related U.S. Application Data
(63) Continuation-in-part of application No. 09/273,520, filed on Mar. 22, 1999.

(51) Int. Cl. A61F 2/06

(52) U.S. Cl. 623/1.11

(57) ABSTRACT

A stent delivery system comprising a catheter including a stent mounting region. A stent is disposed about the stent mounting region of the catheter, the stent having a distal end and a proximal end. The stent further having an unexpanded state and an expanded state, and at least one stent retaining sleeve having a first end and a second end. The first end overlying an end of the stent when the stent is in the unexpanded state, the second end engaged to at least a portion of the catheter adjacent to the stent mounting region. The at least one stent retaining sleeve being at least partially composed of at least one hydrophilic elastomeric material.

### CROSS-REFERENCE TO RELATED APPLICATIONS

1. This application is a Continuation-In-Part application from U.S. application Ser. No. 09/273,520 filed Mar. 22, 1999, the entire contents of which is hereby incorporated by reference.

### STATEMENT REGARDING FEDERALLY SPONSORED RESEARCH

2. Not Applicable

### BACKGROUND OF THE INVENTION

3. 1. Field of the Invention

4. The application relates to a delivery system in which a catheter carries on its distal end portion a stent which is held in place around the catheter prior to and during percutaneous delivery by means of at least one sleeve composed of a hydrophilic elastomer material. The hydrophilic elastomer material provides the sleeve or sleeves with a lubricious surface, thus eliminating or reducing the inclusion of an additional lubricious coating in or on the sleeve material. The stent may be self-expanding, such as a NITINOL shape memory stent, or it may be expandable by means of an expandable portion of the catheter, such as a balloon.

5. 2. Description of the Related Art

6. Stents and stent delivery systems are utilized in a number of medical procedures and situations, and as such their structure and function are well known. A stent is a generally cylindrical prosthesis introduced via a catheter into a lumen of a body vessel in a configuration having a generally reduced diameter and then expanded to the diameter of the vessel. In its expanded configuration, the stent supports and reinforces the vessel walls while maintaining the vessel in an open, unobstructed condition.

7. Both self-expanding and inflation expandable stents are well known and widely available in a variety of designs and configurations. Self-expanding stents must be maintained under a contained sheath or sleeve(s) in order to maintain their reduced diameter configuration during delivery of the stent to its deployment site. Inflation expandable stents are crimped to their reduced diameter about the delivery catheter, then maneuvered to the deployment site and expanded to the vessel diameter by fluid inflation of a balloon positioned between the stent and the delivery catheter. The present invention is particularly concerned with delivery and deployment of inflation expandable stents, although it is generally applicable to self-expanding stents when used with balloon catheters.

8. An example of a stent is described in U.S. Pat. No. 5,972,018, issued Oct. 26, 1999 to Israel et al., the content of which is incorporated herein by reference.

9. In advancing an inflation expandable stent through a body vessel to the deployment site, there are a number of important considerations. The stent must be able to securely maintain its axial position on the delivery catheter, without translocating proximally or distally, and especially without becoming separated from the catheter. The stent, particularly any potentially sharp or jagged edges of its distal and proximal ends, must be protected to prevent edge dissection and prevent abrasion and/or reduce trauma of the vessel walls.

10. Inflation expandable stent delivery and deployment systems are known which utilize restraining means that overlie the stent during delivery. U.S. Pat. No. 4,950,227 to Savin et al., relates to an inflation expandable stent delivery system in which a sleeve overlaps the distal or proximal margin (or both) of the stent during delivery. During inflation of the stent at the deployment site, the stent margins are freed of the protective sleeve(s). U.S. Pat. No. 5,403,341 to Solar, relates to a stent delivery and deployment assembly which uses retaining sheaths positioned about opposite ends of the compressed stent. The retaining sheaths of Solar are adapted to tear under pressure as the stent is radially expanded, thus releasing the stent from engagement with the sheaths. U.S. Pat. No. 5,108,416 to Ryan et al., describes a stent introducer system which uses one or two flexible end caps and an annular socket surrounding the balloon to position the stent during introduction to the deployment site.

11. Copending U.S. patent application Ser. No. 09/407,836 which was filed on Sep. 28, 1999 and entitled Stent Securement Sleeves and Optional Coatings and Methods of Use, and which is incorporated in its entirety herein by reference, also provides for a stent delivery system having sleeves. In Ser. No. 09/407,836 the sleeves may be made up of a combination of polytetrafluoroethylene (PTFE) as well as one or more thermoplastic elastomers. Other references exist which disclose a variety of stent retaining sleeves.

12. A common problem which occurs in catheter assemblies is friction or adhesion between various parts which periodically come into contact with one another during the medical procedure. For instance, friction can occur between the guide catheter and guide wire, between the introducer sheath and the guide catheter, or between the guide catheter and the balloon catheter, for instance, and may increase the difficulty of insertion, cause loss of catheter placement, and result in discomfort to the patient or damage to the vasculature. In catheters equipped with stent retaining socks or sleeves, friction between the balloon and sleeve, and/or the stent and sleeve may also cause retraction of the sleeves to be made more difficult.

13. It is therefore desirable to reduce the friction due to the sliding between the various parts of the catheter assemblies. Copending U.S. application Ser. No. 09/549,286 which was filed Apr. 14, 2000 describes a reduced columnar strength stent retaining sleeve having a plurality of holes. The relatively reduced columnar and radial strength provided by the holes allows the sleeve to be retracted off of a stent without the need for lubricant.

14. Lubricants however may be used in a variety of stent delivery catheters. Many lubricants and lubricious coatings types have been used in conjunction with balloon catheters. Both hydrophilic and hydrophobic coatings and lubricants are well known in the catheter art. For example: copending U.S. patent application Ser. No. 09/427,805 filed Oct. 27, 1999, and entitled End Sleeve Coating for Stent Delivery, describes the use of stent retaining sleeves having lubricious coatings applied thereto.

15. Copending U.S. patent application Ser. No. 09/273,520 filed Mar. 22, 1999, entitled Lubricated Sleeve Material For Stent Delivery likewise describes the use of stent retaining sleeves and lubricants, wherein a lubricant is included in the composition of the stent delivery material.

16. Stent delivery systems which may not require the use of lubricants have been proposed, such as copending U.S. application Ser. No. 09/549,286 mentioned above. Another example of a stent delivery system and retaining sleeve which may not require lubrication is Copending application Ser. No. 09/668,496 filed Sep. 22, 2000 and entitled Striped Sleeve For Stent Delivery describes a two component sleeve having one or more substantially longitudinally oriented stripe of a hard material and a softer material. The striped configuration of materials in the sleeve allows the sleeve to radially expand but with limited or no longitudinal expansion. The unique expansion characteristics provided by the striped configuration helps avoid a need to use a lubricant with the sleeve, though a lubricant may still be utilized therewith if desired.

17. The entire content of all patents and applications listed within the present patent application are incorporated herein by reference.

### BRIEF SUMMARY OF THE INVENTION

18. This invention provides for a stent delivery system which employs one or more stent retaining sleeves composed of a hydrophilic elastomer material. This is in contrast to prior sleeves which are made of non-hydrophilic materials and include hydrophilic coatings on sleeve surfaces as well as the use of lubricants on portions of a stent delivery system.

### BRIEF DESCRIPTION OF THE SEVERAL VIEWS OF THE DRAWINGS

19. A detailed description of the invention is hereafter described with specific reference being made to the drawings in which:

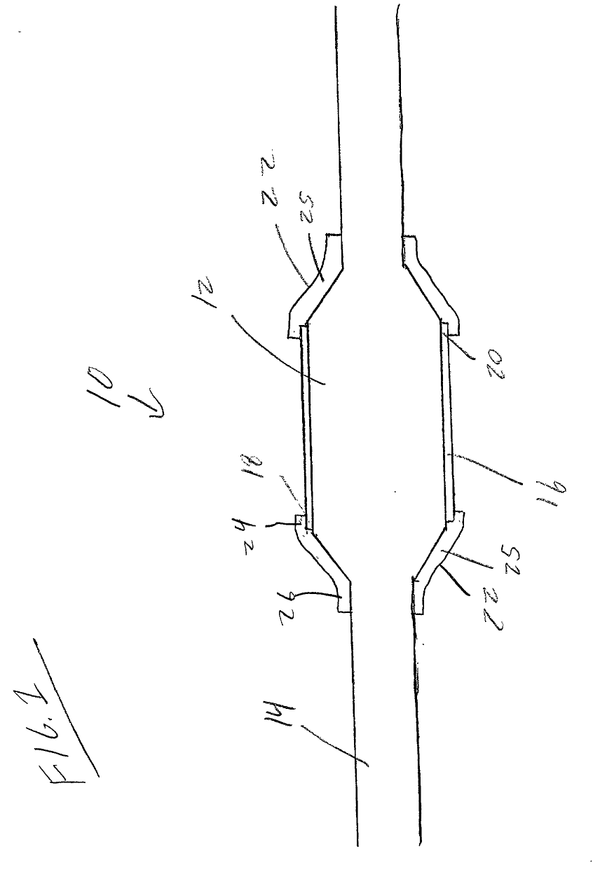

20. FIG. 1 is a perspective view of an embodiment of the invention.

### DETAILED DESCRIPTION OF THE INVENTION

21. As may be seen in FIG. 1, the present invention may be embodied in a stent delivery catheter, indicated generally at 10. Catheter 10 may be fixed wire, rapid exchange or over the wire type. Catheter 10, includes a stent mounting region 12, the stent mounting region 12 may be an inflatable portion of the catheter or may be a separate balloon mounted to the catheter shaft 14. The balloon 12 may have an unexpanded state and an expanded state. A stent 16, disposed about the stent mounting region 12 may be delivered when the balloon 12 is expanded to the expanded state.

22. The stent 16 includes a proximal end 18 and a distal end 20. A stent retaining sleeve 22 overlies at least a portion of each end 18 and 20. As is known in the art, when the balloon 12 and stent 16 are expanded to their expanded state, the ends of the stent retaining sleeves 22 are configured to retract off of the stent ends 18 and 20.

23. In the present invention, the sleeves 22 are composed partially or entirely of a hydrophilic elastomeric material 25. While hydrophilic materials have previously been used to coat or other wise lubricate portions of a stent delivery sleeve, new developments provide for hydrophilic elastomer materials which may be extruded or otherwise used to produce a stent retaining and delivery sleeve 22 such as may be seen in FIG. 1. A specific hydrophilic elastomer material is chosen depending on the material's particular balance of lubriciousness, degree of swelling, wetability and mechanical performance. Hydrophilic elastomer material such as hydrophilic polyurethane compositions have previously been described in U.S. Pat. No. 5,283,298 to Sarpeshkar et al., the entire contents of which are incorporated herein by reference.

24. In the present invention the hydrophilic material 25 may be a commercially available hydrophilic thermoplastic polyurethane composition such as HYDROTHANE™, available from CT Biomaterials, or an aliphatic polyether-based thermoplastic polyurethane such as TECOPHILIC®, available from Thermedics Inc. The particular hydrophilic thermoplastic polyurethane composition may be composed of the copolymers of poly(ethylene oxide) (PEO), poly(N-vinyl-2-pyrrolidone), (PVP) with polyurethanes. Other hydrophilic polymers with good elasticity also may be used. Some examples may be hydrophilic polyester which contains both polyoxyethylene diester and alkylene diesters as copolyester, a hydrophilic polyamide from the block copolymers of nylon and a poly(dioxaamide), such as poly(4, 7-dioxadecamethylene adipamide). These compositions, among others, are described in detail in U.S. Pat. No. 5,239,019 and U.S. Pat. No. 4,235,714, the entire contents of which being respectively incorporated wherein by reference. When used to manufacture sleeves 22 a hydrophilic thermoplastic polyurethane has a durometer hardness of 80A to 72D as measured by the Shore hardness scale.

25. The lubricious nature of the of hydrophilic elastomer material 25 provides the first portion 24 of the sleeve which overlies the stent 16 with a reduced frictional engagement with the stent 16. The improved lubricity of the material 25 also provides the sleeve 22 as well as the catheter 10 with improved trackability through a vessel or body lumen.

26. The second portion 26 of the sleeve 22 is disposed about and is engaged to a portion of the catheter shaft 14 adjacent to the balloon 12.

27. As stent 16 is expanded, the stent ends 18 and 20 will eventually be drawn from underneath the stent retaining sleeves 22 and the sleeves 22 will retract.

28. It should be noted that a stent retaining sleeve 22 composed of a hydrophilic elastomeric material 25 may be embodied in a variety of sleeve types and forms. For example: U.S. Pat. No. 5,788,707, incorporated herein by reference, describes a pull back sleeve, such a pull back sleeve could be manufactured from a hydrophilic elastomer material such as is described herein. Likewise, copending application Ser. No. 09/664,268, filed Sep. 18, 2000, and incorporated herein by reference, describes a rolling sleeve. Such a rolling sleeve may also be composed in whole or in part of the hydrophilic elastomeric sleeve material of the present invention. Numerous configurations of sheathes, sleeves and socks are known in the stent delivery and catheter art. The present invention is also directed to the use of the present hydrophilic elastomer material in constructing these devices.

29. In addition to the above, it should also be noted that the sleeves 22 of the present invention may also be utilized with any of the variety of coatings such as are known.

30. In addition to being directed to the embodiments described above and claimed below, the present invention is further directed to embodiments having different combinations of the features described above and claimed below. As such, the invention is also directed to other embodiments having any other possible combination of the dependent features claimed below.

31. The above examples and disclosure are intended to be illustrative and not exhaustive. These examples and description will suggest many variations and alternatives to one of ordinary skill in this art. All these alternatives and variations are intended to be included within the scope of the attached claims. Those familiar with the art may recognize other equivalents to the specific embodiments described herein which equivalents are also intended to be encompassed by the claims attached hereto.

1. A stent delivery system comprising:
a catheter including a stent mounting region;
a stent disposed about the stent mounting region of the catheter, the stent having a distal end and a proximal end, the stent further having an unexpanded state and an expanded state, and
at least one stent retaining sleeve having a first end and a second end,
the first end overlying an end of the stent when the stent is in the unexpanded state, the second end engaged to at least a portion of the catheter adjacent to the stent mounting region;
the at least one stent retaining sleeve being at least partially composed of at least one hydrophilic elastomeric material.

2. The stent delivery catheter of claim 1 wherein the at least one hydrophilic elastomeric material is TECOPHILIC.

3. The stent delivery catheter of claim 1 wherein the at least one hydrophilic elastomeric material is HYDROTHANE.

4. The stent delivery catheter of claim 1 wherein the at least one hydrophilic elastomeric material is at least partially comprised of hydrophilic polyurethane.

5. The stent delivery catheter of claim 1 wherein the at least one hydrophilic elastomeric material is at least partially comprised of hydrophilic polyesters.

6. The stent delivery catheter of claim 1 wherein the at least one hydrophilic elastomeric material is at least partially comprised of hydrophilic polyamides.

7. The stent delivery catheter of claim 1 where in the at least one hydrophilic elastomeric material has a predetermined hardness, the predetermined hardness being selected from a durometer value between about 80A-72D.

8. A stent retaining sleeve for retaining in an expanded state at least one end of a stent on a balloon catheter comprising a tubular member having a first end and a second end:
the first end constructed and arranged to overlay an end of a stent, the second end constructed and arranged to be in contact with at least a portion of a catheter adjacent to a stent mounting region;
at least a portion of the tubular member composed of at least one hydrophilic elastomer material.

9. A stent delivery system comprising:
a catheter including a stent mounting region;
a stent disposed about the stent mounting region of the catheter, the stent having a distal end and a proximal end, the stent further having an unexpanded state and an expanded state, and
at least one hydrophilic elastomeric stent retaining sleeve having a first end and a second end,
the first end overlying an end of the stent when the stent is in the unexpanded state, the second end engaged to at least a portion of the catheter adjacent to the stent mounting region.
